(19) United States
(12) Patent Application Publication
(10) Pub. No.: US 2016/0353979 A1
(43) Pub. Date: Dec. 8, 2016

(54) BENDABLE TREATMENT INSTRUMENT

(71) Applicant: KYUSHU UNIVERSITY, NATIONAL UNIVERSITY CORPORATION, Fukuoka-shi, Fukuoka (JP)

(72) Inventors: Makoto HASHIZUME, Fukuoka (JP); Hajime KENMOTSU, Fukuoka (JP); Ryu NAKADATE, Fukuoka (JP)

(73) Assignee: KYUSHU UNIVERSITY, NATIONAL UNIVERSITY CORPORATION, Fukuoka-shi, Fukuoka (JP)

(21) Appl. No.: 15/103,249

(22) PCT Filed: Dec. 12, 2014
(86) PCT No.: PCT/JP2014/082951

§ 371 Date: Jun. 9, 2016

(30) Foreign Application Priority Data
Dec. 13, 2013 (JP) 2013-258646

(51) Int. Cl. A61B 1/00; A61B 90/50; A61B 34/00; A61B 17/29; A61B 17/32

(52) U.S. Cl. CPC A61B 1/00133; A61B 17/2909; A61B 17/320068; A61B 34/70; A61B 90/50; A61B 2017/00336

(57) ABSTRACT

Provided is a bendable treatment instrument that enables an individual doctor who is performing an endoscopic procedure to reliably and intuitively manipulate forceps, a knife, or the like. A bendable treatment instrument (1) includes a mechanism connecting, via a flexible tube body (4) encasing a transmission member, a bending section (3) configured to perform a bending action and a manipulation member (8) configured to receive a manipulation with respect to the bending section (3) from an operator. The mechanism is capable of transmitting a manipulation by the manipulation member (8) to the bending section (3). The bendable treatment instrument further includes a support member (7) configured to support the manipulation member (8) while clasping the flexible tube body (4), and a rotation support member (11) configured to support the manipulation member (8) so as to allow the manipulation member (8) to axially rotate with respect to the flexible tube body (4) at the support member (7).

### TECHNICAL FIELD

The present invention relates to a bendable treatment instrument integrated with forceps, a high-frequency knife, or the like, that is used by being set in an endoscope for performing a procedure on an intra-abdominal organ or a hollow organ, such as the gastrointestinal tract, of the human body. More particularly, the present invention relates to a bendable treatment instrument that enables an individual doctor to reliably and intuitively manipulate forceps, a high-frequency knife, or the like.

### BACKGROUND ART

In recent years, when treating diseases of a hollow organ, such as the gastrointestinal tract, of the human body, if the disease is, e.g., early stomach cancer where the tumor is confined to the mucosal lining, it has become standard practice to use an endoscope to perform an excision by using forceps or a knife protruding from a hole provided in the endoscope, instead of performing a laparotomy or surgery using a laparoscope as was performed in the past.

Further, in the last few years, in order to minimize invasiveness to the body, a new procedure (NOTES: Natural Orifice Translumenal Endoscopic Surgery) that avoids leaving an incision mark on the body surface by using a natural opening in the body, such as the mouth, the anus, or, in the case of women, the vagina, as an entry portal for inserting an endoscope, making a small incision in the lumen wall of these openings to allow the endoscope to reach the abdominal cavity, and then performing diagnosis, or using the above-mentioned forceps or the like, has also begun to be performed in intra-abdominal surgery normally performed by laparotomy of by using a laparoscope.

As an example of a manipulation instrument for the forceps or the high-frequency knife to be used in such an endoscopic procedure, a bendable tube including a bending section capable of bending has been proposed (refer to Patent Literature 1). This bendable tube includes: a main body having a tubular cavity; a pair of transmission members, each of which is inserted into the bending section and each of which has a tip end that is connected to a tip end of the bending section or to the main body closer to the tip end side than the bending section; and a manipulation member to which a base end of the pair of transmission members is connected, and that is configured such that, when one of the pair of transmission members is retracted toward the base end side, the other one of the pair of transmission members is pushed out toward the tip end side in cooperation therewith. In this bendable tube, a predetermined magnitude of pretension is applied to the pair of transmission members by the retracting action.

Also proposed is an endoscopic surgical instrument that is a narrow-diameter flexible tube including, on a tip end side, a bending section capable of bending in a single plane (refer to Patent Literature 2). In this endoscopic surgical instrument, a plane of movement of a manipulation member connected to a base end side of the flexible tube and a plane of bending of the bendable section correspond to each other on a one-to-one basis.

Also proposed is a manipulation system capable of simultaneously manipulating forward/back movement and multiple degrees of bending freedom of a bending section, which is connected to a tip end side of a similarly elongate bendable tube and which has multiple degrees of bending freedom (refer to Patent Literature 3).

### CITATION LIST

### Patent Literature

Patent Literature 1: Japanese Patent Application Laid-open No. 2009-279405

Patent Literature 2: U.S. Pat. No. 8,137,263 B2

Patent Literature 3: Japanese Translation of PCT International Application No. 2010-511440

### SUMMARY OF INVENTION

### Technical Problem

The above-mentioned endoscopes are provided with a hole (referred to as a treatment instrument insertion channel, which has, for example, an inner diameter of 2.8 mm and a length of 1 to 1.5 m) through which the treatment instrument, such as forceps or a knife, is inserted. When using the treatment instrument during a surgical procedure, the treatment instrument is set by inserting the treatment instrument into the treatment instrument insertion channel. A bending section is provided at a tip end portion of the treatment instrument. In order to achieve intuitive manipulation by applying an input on a manipulation member located at a base end portion, the following functions need to be provided.

Function 1. Have at least three degrees of freedom in addition to the actions of an electric scalpel or a clasping instrument. In order for the tip end of the bending section to reach an arbitrary point in space, a total of three degrees of freedom, namely, forward/back movement, a twisting action, and a bending action, are necessary.

Function 2. The manipulation member should be shaped so that input directions are intuitively understood, for example, should have a shape like a joystick. Further, it is desired that the manipulation direction applied to the manipulation member spatially match the bending direction of the bending section. In order to achieve this, it is necessary for the manipulation member to be fixed to the floor or the like so that the orientation of the manipulation member does not change.

Although the related-art discussed in Patent Literature 1 does allow three-dimensional actions of the forceps and the like based on wire manipulations, the related-art discussed in Patent Literature 1 does not envisage a situation in which the treatment instrument is used by being inserted into the above-mentioned endoscope hole.

On the other hand, the related-art discussed in Patent Literature 2 does envisage a situation in which the treatment instrument is used by being inserted into the treatment instrument insertion channel of the endoscope. Further, in this technology, because the treatment instrument has a total of three degrees of freedom—i.e., one degree of freedom of forward/back movement of an insertion member (pulling the treatment instrument in and out), one degree of freedom of twisting rotation of the insertion member, and one degree of freedom of bending of the bending section—the treatment instrument according to Patent Literature 2 has the above-mentioned Function 1, and is capable of reaching an arbitrary position in space. However, when a flexible tube sufficiently elongate to be inserted into the treatment instrument insertion channel is employed, the twisting rotation is not completely transmitted due to friction at an inner surface of the treatment instrument insertion channel, causing deviation to occur. As a result of this deviation, the manipulation plane of the manipulation member and the bending plane of the bending section are constantly changing, and hence a state in which those planes spatially match does not last. Therefore, in the related-art discussed in Patent Literature 2, the above-mentioned Function 2 is not satisfied, and the treatment instrument cannot be intuitively manipulated. Thus, rather than combining twisting rotation with bending in a single plane, intuitive manipulation is more likely to be achieved by combining two instances of bending in a single plane (bending up/down and left/right) without using twisting rotation.

Further, the related-art discussed in Patent Literature 3 also envisages a situation in which the treatment instrument is used by being inserted into the treatment instrument insertion channel of the endoscope, and envisages a bending section having, in addition to a degree of freedom of forward/back movement of the bendable tube (pulling the treatment instrument in and out), degrees of freedom of bending in the up/down and left/right directions. Therefore, the related-art discussed in Patent Literature 3 has a minimum of three degrees of freedom, and hence satisfies the above-mentioned function 1. Patent Literature 3 also proposes a manipulation member that is fixed to the ground, and that has a shape close to that of a joystick. However, when inserting the treatment instrument into the treatment instrument insertion channel, the insertion member can get twisted by the friction at the inner surface of the treatment instrument insertion channel and by the path in the treatment instrument insertion channel Unless this twisting is resolved, deviation is produced between the bending direction of the bending section and the input direction of the manipulation member. If even a slight amount of this deviation remains, intuitive manipulation of the treatment instrument cannot be achieved. Therefore, Patent Literature 3 does not satisfy the above-mentioned Function 2.

The present invention has been created in view of problems such as those described above. An objective of the present invention is to provide a bendable treatment instrument that enables an individual doctor performing an endoscopic procedure to reliably and intuitively manipulate forceps, a high-frequency knife, or the like.

### Solution to Problem

In order to solve problems such as those described above, the present invention employs the following means.

Specifically, the invention according to claim 1 relates to a bendable treatment instrument including a mechanism that connects, via a flexible tube body encasing a transmission member, a bending section configured to perform a bending action and a manipulation member configured to receive a manipulation with respect to the bending section from an operator, the mechanism being capable of transmitting a manipulation by the manipulation member to the bending section, the bendable treatment instrument further including a support member configured to support the manipulation member while clasping the flexible tube body, and a rotation support member configured to support the manipulation member so as to allow the manipulation member to axially rotate with respect to the flexible tube body at the support member. Further, it is preferred that the bendable treatment instrument further include a lock member configured to inhibit or to permit axial rotation of the manipulation member at the rotation support member.

According to the bendable treatment instrument of the present invention, even if deviation occurs between the bending direction of the bending section that appears in the imaging screen of the endoscope and the direction of manipulation at the doctor's hands by the manipulation member due to the flexible tube body getting twisted, for example, this deviation can be resolved by releasing a lock mechanism of the lock member and axially rotating the manipulation member as appropriate at the rotation support member independently of the flexible tube body. Then, by again setting the lock mechanism of the lock member, it is possible to secure and maintain a state in which the manipulation feeling at the manipulation member matches the behavior, etc., of the operating member shown by the endoscope image. Therefore, the bendable treatment instrument according to the present invention enables an individual doctor who is performing an endoscopic procedure to reliably and intuitively manipulate forceps, a knife, or the like.

Note that, in the bendable treatment instrument described above, it is preferred that the support member is configured to stand upright from a predetermined base. According to such a bendable treatment instrument, because the support member is stably fixed, the axial direction of the manipulation member is fixed. As a result, after the above-described deviation has been resolved and the lock reset, intuitive manipulation can be stably continued. Further, fixing the endoscope to the predetermined base allows the operator to release his or her hands from the manipulation member, so that the endoscope can be manipulated as necessary. As a result, the procedure can be performed even by an individual doctor, thus enabling large improvement in the efficiency of the procedure compared with the past.

Further, it is preferred that the support member include a sliding mechanism configured to slide on the predetermined base. In addition, it is preferred that the flexible tube body be encased within an outer tube fixed to the base. In such a configuration, it is more preferred that one end of the outer tube be clasped at the predetermined base, and the other end be clasped at an entrance of a treatment instrument insertion channel of an endoscope or at a tip end of an endoscope. According to such a bendable treatment instrument, when the mechanism of the operating member is manipulated in a manner that moves the flexible tube body forward, namely, be paid out in the direction of the operating member, such as forceps or a knife, the support member clasping the flexible tube body slides forward, enabling the operating member to be freely advanced and retracted while still allowing the doctor to intuitively manipulate the operating member.

Further, in the above-mentioned bendable treatment instrument, forceps or a high-frequency knife may be provided at a tip end of the bending section. In addition, it is more preferred that the flexible tube body has an outer diameter that is smaller than 2.8 mm. According to such a bendable treatment instrument, the flexible tube body is set via a small-diameter hole of about 2.8 mm provided in the endoscope, thus enabling the operating member, such as forceps or a high-frequency knife, to be used in a surgical procedure.

### Advantageous Effects of Invention

As described above, the bendable treatment instrument according to the present invention enables an individual doctor who is performing an endoscopic procedure to reliably and intuitively manipulate forceps, a knife, or the like.

### BRIEF DESCRIPTION OF DRAWINGS

FIG. 1 is an explanatory diagram of an endoscopic therapy mechanism including a bendable treatment instrument according to an embodiment.

FIG. 2 is a perspective diagram illustrating a configuration example of the bendable treatment instrument according to the embodiment.

### DESCRIPTION OF EMBODIMENT

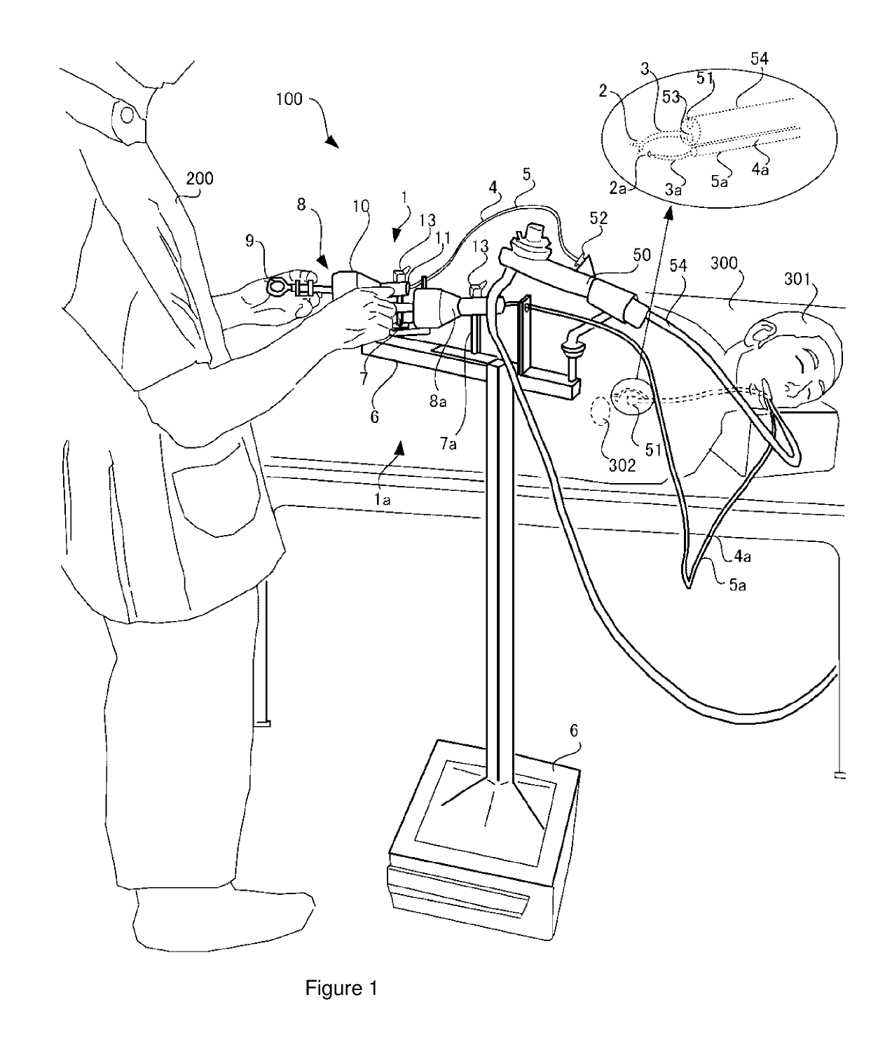

An embodiment of the present invention is now described with reference to the drawings. FIG. 1 is an explanatory diagram illustrating a configuration example of an endoscopic therapy mechanism 100 including an endoscope and one or a plurality of bendable treatment instruments according to this embodiment. In this example, an embodiment in which two bendable treatment instruments are used is described. The bendable treatment instruments 1 and 1a according to this embodiment include, for example, forceps, or a high-frequency knife, as operating members 2 and 2a to be used with an endoscope system 50 in a surgical procedure, for a low-invasive endoscopic procedure performed on a disease of the gastrointestinal tract, which is a hollow organ, or the like. The operating members 2 and 2a are provided at a tip end of respective bending sections 3 and 3a. The operating members 2 and 2a and the bending sections 3 and 3a are treatment instruments controlled by an operator 200, such as a doctor, based on images captured by a camera 51 included in the endoscope system 50. The operating members 2 and 2a and the bending sections 3 and 3a are provided at a tip end of respective flexible tube bodies 4 and 4a. The flexible tube body 4 is set with respect to a base 6, and is configured so that one end of the flexible tube body 4 is inserted into a treatment instrument insertion channel 52 of the endoscope system 50 by being passed through a lumen of an outer tube 5—whose distal end is fixed to the treatment instrument insertion channel 52 of the endoscope system 50—and reaches an exit 53 of the treatment instrument insertion channel 52, which is provided close to the camera 51. An ordinary endoscope system only has one treatment instrument insertion channel; so, the second flexible tube body 4a is set with respect to the base 6, and is configured so that one end of the flexible tube body 4a is passed through a lumen of an outer tube 5a—whose distal end is fixed to a tip end of the endoscope system 50—and reaches the vicinity of the camera 51. In this case, the operator 200 causes the camera 51 and the operating members 2 and 2a to reach a treatment site 302 by inserting an insertion member 54 of the endoscope—with the camera 51 at the front—and the outer tube 5a integrated therewith into, for example, the oral cavity of a patient 301 who is lying on a bed 300.

On the other hand, the posterior end of each of the flexible tube bodies 4 and 4a is fixed to respective support members 7 and 7a, which are standing upright from the predetermined base 6. Manipulation members 8 and 8a, which receive manipulations with respect to the above-mentioned operating members 2 and 2a and bending sections 3 and 3a from the operator 200, are supported by the respective support members 7 and 7a standing upright from the predetermined base 6 while clasping the respective flexible tube bodies 4 and 4a. Note that, as illustrated in FIG. 1, the base 6 from which the support members 7 and 7a stand upright also supports the endoscope system 50 at the same time.

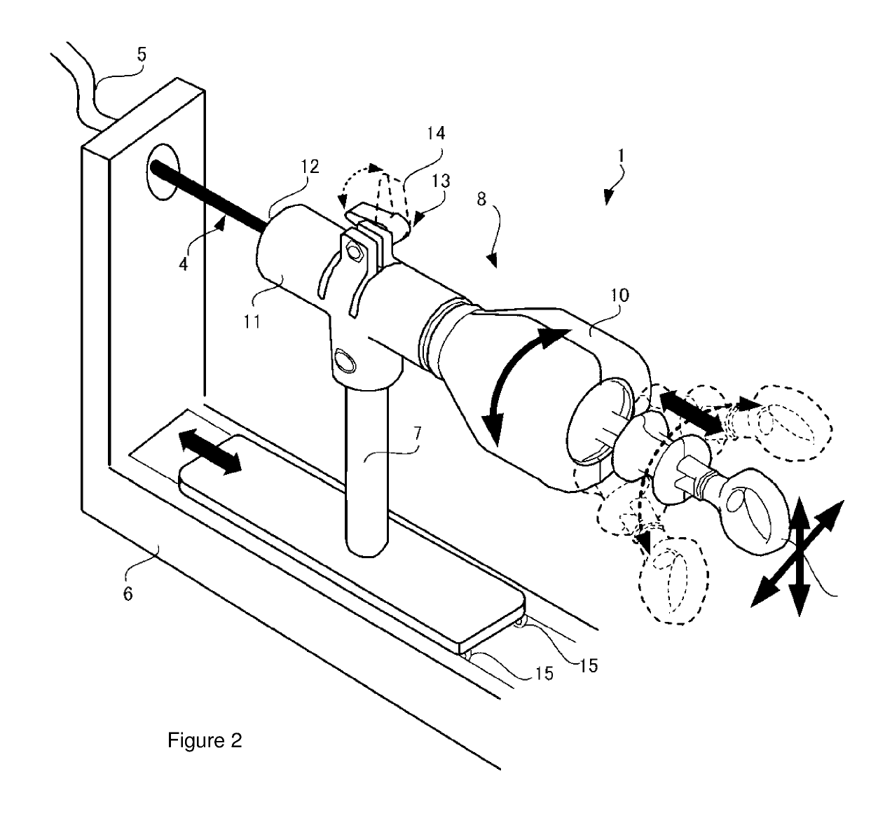

Next, the configuration of the bendable treatment instrument 1 is described in more detail. FIG. 2 is a perspective diagram illustrating a configuration example of the bendable treatment instrument 1 according to this embodiment. A case 10 in the manipulation member 8 is clasped by a rotation support member 11 which encases the case 10 so as to permit axial rotation thereof. On the other hand, the rotation support member 11 is configured to clasp, at its tip end portion 12, the flexible tube body 4 so as not to permit axial rotation of the flexible tube body 4. This rotation support member 11 is a part of the support member 7, and includes a lock mechanism, namely, a lock member 13, configured to inhibit axial rotation of the case 10 by suitably pressing against the outer surface of the case 10, which is encased by the rotation support member 11. Although an illustration of a specific example of the mechanism is not provided, an example of a mechanism that may be used is a mechanism configured so that, when a lever 14 of the lock member 13 is turned from a horizontal state (state shown by a solid line in FIG. 2) to a vertical state (state shown by a solid line in FIG. 2), the turning of the lever 14 causes a predetermined protrusion to be lowered in a downward direction from an upper portion of the hollow interior of the rotation support member 11, and to press down on the outer surface of the case 10 that is being clasped. Obviously, other known mechanisms may be employed as appropriate.

Note that the support member 7 including the above-mentioned rotation support member 11 has a configuration that allows it to slide on the predetermined base 6 via a sliding mechanism 15, such as a pulley. In this case, when the operator 200 pushes a grip portion 9 of the manipulation member 8 in the direction of the operating member 2, namely, in a forward direction, the flexible tube body 4 is pressed forward with respect to the outer tube 5. As a result, the operating member 2 and the bending section 3 can be freely advanced and retracted while still being intuitively manipulated by the doctor.

Thus, according to the bendable treatment instrument of the present invention, an individual doctor who is performing an endoscopic procedure can reliably and intuitively manipulate forceps, a knife, or the like.

### REFERENCE SIGNS LIST

1, 1a: Bendable treatment instrument

2, 2a: Operating member

3, 3a: Bending section

4, 4a: Flexible tube body

5, 5a: Outer tube

6: Base

7, 7a: Support member

8, 8a: Manipulation member

9: Grip portion

10: Case

11: Rotation support member

12: Support member's tip end

13: Lock member

14: Lever

15: Sliding mechanism

50: Endoscope system

51: Camera

52: Treatment instrument insertion channel

53: Treatment instrument insertion channel's exit

54: Endoscope's insertion member

100: Endoscopic therapy mechanism

200: Operator

300: Bed

301: Patient

302: Treatment site

1. A bendable treatment instrument, comprising a mechanism connecting, via a flexible tube body encasing a transmission member, a bending section configured to perform a bending action and a manipulation member configured to receive a manipulation with respect to the bending section from an operator, the mechanism being capable of transmitting a manipulation by the manipulation member to the bending section, the bendable treatment instrument further comprising:
a support member configured to support the manipulation member while clasping the flexible tube body; and
a rotation support member configured to support the manipulation member with respect to an axis of the flexible tube body at the support member so as to allow an axis of the manipulation member to axially rotate about the axis of the flexible tube body.

2. The bendable treatment instrument according to claim 1, further comprising a lock member configured to inhibit or to permit axial rotation of the manipulation member at the rotation support member.

3. The bendable treatment instrument according to claim 1, wherein the support member is configured to stand upright from a predetermined base.

4. The bendable treatment instrument according to claim 1, wherein the support member comprises a sliding mechanism configured to slide on the predetermined base.

5. The bendable treatment instrument according to claim 1, wherein the flexible tube body is encased in an outer tube fixed to the base.

6. The bendable treatment instrument according to claim 1, wherein forceps or a high-frequency knife is provided at a tip end of the bending section.

7. The bendable treatment instrument according to claim 1, wherein the flexible tube body has an outer diameter that is smaller than 2.8 mm.

8. The bendable treatment instrument according to claim 2, wherein the support member is configured to stand upright from a predetermined base.

9. The bendable treatment instrument according to claim 2, wherein the support member comprises a sliding mechanism configured to slide on the predetermined base.

10. The bendable treatment instrument according to claim 2, wherein the flexible tube body is encased in an outer tube fixed to the base.

11. A bendable treatment instrument, comprising a mechanism connecting, via a flexible tube body encasing a transmission member, a bending section configured to perform a bending action and a manipulation member configured to receive a manipulation with respect to the bending section from an operator, the mechanism being capable of transmitting a manipulation by the manipulation member to the bending section, the bendable treatment instrument further comprising:
a support member configured to support the manipulation member; and
a rotation support member configured to support the manipulation member at the support member so as to allow an axis of the manipulation member to axially rotate about an axis of the flexible tube body.

12. The bendable treatment instrument according to claim 11, further comprising a lock member configured to inhibit or to permit axial rotation of the manipulation member at the rotation support member.

13. The bendable treatment instrument according to claim 11, wherein the support member is configured to stand upright from a predetermined base.

14. The bendable treatment instrument according to claim 11, wherein the support member comprises a sliding mechanism configured to slide on the predetermined base.

15. The bendable treatment instrument according to claim 11, wherein the flexible tube body is encased in an outer tube fixed to the base.

16. The bendable treatment instrument according to claim 11, wherein forceps or a high-frequency knife is provided at a tip end of the bending section.

17. The bendable treatment instrument according to claim 11, wherein the flexible tube body has an outer diameter that is smaller than 2.8 mm.

18. The bendable treatment instrument according to claim 12, wherein the support member is configured to stand upright from a predetermined base.

19. The bendable treatment instrument according to claim 12, wherein the support member comprises a sliding mechanism configured to slide on the predetermined base.

20. The bendable treatment instrument according to claim 12, wherein the flexible tube body is encased in an outer tube fixed to the base.
